(19) United States
(12) Patent Application Publication
(10) Pub. No.: US 2005/0090888 A1
(43) Pub. Date: Apr. 28, 2005

(54) Pleated stent assembly

(76) Inventors: Richard A. Hines, Stilwell, KS (US); Ben Z. Roitberg, Lincolnwood, IL (US)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 10/695,527

(22) Filed: Oct. 28, 2003

(51) Int. Cl. A61F 2/06

(52) U.S. Cl. 623/1.11; 623/1.15; 623/1.29

(57) ABSTRACT

The present invention is directed to a pleated medical device assembly, preferably a pleated stent assembly, comprising a tube co-pleated with a balloon to a delivery width suitable for intraluminal delivery. Because the tube of the present invention transitions between its original diameter and its delivery diameter by folding and unfolding, rather than by radial contraction and expansion, the wall of the tube of the present invention may be substantially non-expandable, and thus may be substantially solid. The pleated stent assembly of the present invention is particularly suited for the treatment of neurovascular aneurysms.

### CROSS-REFERENCE TO RELATED APPLICATIONS

Not Applicable.

### STATEMENT REGARDING FEDERALLY SPONSORED RESEARCH OR DEVELOPMENT

Not Applicable.

### BACKGROUND OF THE INVENTION

The present invention is directed to the field of stenting and more specifically to the field of medical and veterinary stents for endovascular treatments.

A stent is a tubular medical device typically inserted into the lumen of a vessel, or other organ, to open the vessel and/or maintain the vessel in an open position to maintain flow within the vessel. Stents are typically introduced to the body percutaneously and delivered intraluminally, via a catheter, to a desired position in the lumen of the vessel.

Stents are generally cylindrical shells comprised of interconnected elements or struts. The pattern of struts on the surface of the cylinder allows the stent to be crimped to a small diameter for delivery and to expand radially from the small delivery diameter to a larger placement diameter once positioned with the lumen. The final placement diameter of the expandable stents is generally between 2.5 and 4 times the delivery diameter. As a result, the surface of the expanded stent has a significant amount of open space. At the small delivery diameter, the metal struts of the stents cover about 50 percent of the surface area of the stent. At the expanded placement diameter, the area covered by the struts is only about 12 to 20 percent of the stent wall.

Stents can be balloon-expandable or self-expanding. The balloon-expandable stent is crimped around the pleated balloon of a balloon catheter to form a small diameter cylinder that can be delivered intraluminally and expanded radially by the expanding balloon. Plastic deformation of the stent struts during balloon expansion results in a final placement diameter sufficient to contact the lumen wall. The final placement diameter is larger than the original pre-crimped diameter. Self-expanding stents, which are formed of elastic material, are elastically compressed from their as-manufactured placement diameter and placed into a sleeve on the distal end of a catheter. Once the stent is in place in the vessel, the stent is pushed out of the sleeve and the stent expands radially to its original pre-compressed diameter without use of a balloon.

Conventional stents expanded from a small cylinder to a large cylinder on a typical balloon are non-uniformly expanded due to non-uniform tension in the stent struts generated as the balloon expands by unpleating. The typical balloon is a non-compliant balloon, pleated along longitudinal pleat lines. The non-uniform expansion results from the fundamental mismatch between the manner in which the non-compliant balloon expands and the manner in which the stent expands. The balloon expands by unfolding, whereas the stent expands by stretching of its circumference. The stent stretching is accomplished by bending of struts that transforms some of the longitudinal components of a strut to a circumferential component, allowing the circumference of the stent to expand.

Friction between the stent struts and the balloon surface results in non-uniform expansion of the stent. A non-compliant balloon is pleated so that only a fraction of the balloon's surface is at the surface when the stent is crimped onto the balloon at the delivery diameter. By contrast, all of the stent is located in a cylindrical shell. At the pleat line, new balloon material is pulled to the surface of the expanding cylinder. The struts over the longitudinal pleat line are loaded circumferentially in tension as the balloon material on each side of the pleat line moves away from the pleat line during expansion. The tension in the struts decreases as the circumferential distance form the pleat line increases. The tension results in sliding of the stent struts relative to the balloon surface.

The amount of sliding and deformation of the stent pattern is largest near the longitudinal line over the pleat. The high tension over the pleat line results in widely spaced struts in the stent surface that was over the pleat line and closely spaced struts in areas that areas midway between pleat lines. Thus, a tri-fold balloon tends to result in an expanded stent with a longitudinal pattern of closely spaced struts and widely spaced struts that is repeated three times around the circumference of a stent. Non-uniform expansion weakens the stent and increases the size of the openings between the stent struts that may allow tissue of the vessel to prolapse.

Stent placement surgery is minimally invasive and is efficacious for some types of vascular diseases. For example, stenting has proven to be effective in the treatment of coronary artery disease. As doctors and researchers attempt to bring the benefits of stenting to other body vessels, currently available stents are sometimes not adequate for the new applications. For example, although use of stents to treat neurovascular aneurysms has been considered, stents currently available are not suitable. Substantial open spaces in the walls of expandable stents do not sufficiently cover the aneurysm to block blood flow to the aneurysm. Solid stents do not have a variable diameter such that there is a high likelihood that the stent would be too large and harm the fragile vessel, or be too small and migrate through the vessel. Solid stents also do not have the flexibility required for delivery through the carotid siphon to the neurovascular arteries. Thus, standard stents have not been successful in treating neurovascular aneurysms.

Neurovascular arterial aneurysm rupture is the most common cause of spontaneous subarachnoid hemorrhage and one of the most common and severe diseases treated by neurosurgeons. Aneurysms can form in various locations along the arterial tree, but are most common at the base of the skull, in the arterial structure known as the “Circle of Willis”. The most common neurovascular aneurysm shape is that of a round bag, or a berry. Such aneurysms are sometimes called “berry aneurysms” or saccular aneurysms. Another type of aneurysm found both in neurovascular arteries and other arteries is the fusiform aneurysm, which is an elongated spindle-shaped dilation of an artery.

Neurovascular aneurysms can become symptomatic in various ways. The most devastating aneurysm outcome is acute rupture, which causes bleeding either around the brain, called “subarachnoid hemorrhage,” or less often, into the brain tissue itself About a third of the patients die before getting medical attention, and many others die despite treatment. Among the survivors of acute aneurysm rupture, permanent neurological damage is common. The damage is caused by the initial injury to the brain, as well as delayed complications. A common and devastating complication is vasospasm—a narrowing of the cerebral arteries that is believed to be caused by a reaction to blood products in the subarachnoid space.

After a neurovascular aneurysm ruptures, it has an elevated risk of rupturing again—about 50 percent in the first six months, mostly within the initial days or weeks after the first event. Therefore, aneurysms in survivors of acute subarachnoid hemorrhage have to be treated urgently to prevent repeat rupture and a very high risk of death.

In addition to bleeding, neurovascular aneurysms can expand without rupture and cause symptoms by exerting pressure on neural structures. Although it is sometimes possible to identify aneurysms that are at risk before rupture occurs and treat them preventively, this subject is controversial. It is difficult, if not impossible, to determine if an unruptured aneurysm will in fact rupture, and the balance between the risk of intervention itself, compared to the risk of rupture if the aneurysm is left alone, is unclear. There is a general opinion that larger aneurysms, more than 5 mm in diameter, and definitely those larger than 10 mm, should be treated prophylactically. If a safer method to treat aneurysms is found, the indications for prophylactic treatment can expand significantly.

Currently, two methods to treat neurovascular aneurysms are approved by the FDA. The first involves a craniotomy and clipping of the aneurysm. This is an open surgical procedure, wherein the arteries are exposed and one or more clips are applied across the neck of the aneurysm to stop blood from flowing into the aneurysm. Clipping the aneurysm is believed to permanently exclude it from the circulation. The dome of the aneurysm can be emptied of blood and collapsed, allowing treatment of the mass effect. Although many advances have occurred over the past thirty years to improve the efficacy and safety of the procedure, there remains a risk associated with the craniotomy itself. The stress and risk of a major surgical procedure is exacerbated in patients with a recent brain injury and in the elderly or medically complicated patients. Brain exposure and retraction may cause further injury to the brain and additional neurological defect. Therefore, less invasive methods for treatment of aneurysms are attractive.

A second, less invasive procedure approved by the FDA involves the insertion of Guiglielmi detachable coils using intraarterial angiography. A pre-formed platinum coil is advanced into the aneurysm via a catheter and is detached using an electric current. Additional coils are then advanced to fill the aneurysm cavity. The coils induce thrombosis. Ultimately, the aneurysm with tightly packed coils has no blood flow within it and is excluded from the circulation.

There are limitations to the aneurysm coiling technique. Coiling of aneurysms is a technically demanding procedure, with a long learning process. In addition, the method works best for round aneurysms with a small neck. In other aneurysms, the packing of the aneurysm is more difficult, and either the neck is left open, or coils protrude into the parent vessel with attendant risk of clot formation and embolism. Balloons or balloon-stent combinations are now used to help pack the aneurysm and keep the coils inside. Despite such advances, the long-term efficacy of aneurysm coiling remains uncertain. Long-term follow up of coiled aneurysms often reveals a recurrent aneurysm neck, and a rupture rate of about 1 percent per year.

### BRIEF SUMMARY OF THE INVENTION

The present invention is directed to a pleated medical device assembly, preferably a pleated stent assembly, comprising a tube co-pleated with a balloon to a delivery width suitable for intraluminal delivery. In use, the assembly is inserted into a body vessel and positioned at a target location within the lumen of a vessel. Once at the target location, the pressure in the balloon is increased to fully unfold the balloon and the tube to their original diameters. The pressure in the balloon is further increased to expand at least a portion of the tube until at least a portion of the exterior surface of the tube presses against the interior of the vessel to hold the tube in place within the vessel. The pressure in the balloon is then released to deflate the balloon, which is then removed from the tube, the vessel and the body.

The tube wall is preferably comprised of a pattern of interconnected solid areas defining open spaces therebetween. Because the tube of the present invention transitions between its original diameter and its delivery width by folding and unfolding, rather than by radial contraction and expansion, the wall of the tube of the present invention may be substantially solid.

In one embodiment, the wall of the tube comprises an annular body section and an annular anchor section. The pattern of the tube is designed to restrict radial expansion substantially beyond the original diameter of the tube within the body section of the tube and to allow radial expansion of the tube beyond the original diameter of the tube within the anchor section. Thus, after the tube is unpleated within the vessel, and the balloon pressure is further increased, only the anchor section expands beyond the original diameter of the tube to anchor the tube in place within the vessel. In such embodiment, the pattern in the body section of the wall preferably comprises greater than about 60 percent solid area.

In the preferred embodiment, the medical device of the pleated medical device assembly is a stent. Use of the pleated stent assembly of the present invention has many advantages. The stent of the present invention is co-pleated with the balloon and unpleated with the balloon as the balloon expands within the vessel, essentially eliminating non-uniform stent expansion. Minor subsequent expansion of the stent to fine tune its diameter to properly fit the stent within the lumen is accomplished by expanding both the balloon and the stent with additional pressure in the balloon. Thus, during both the unfolding and fine-tune expanding, there is no tendency for non-uniform expansion.

The pleated stent assembly of the present invention may be used in conventional stenting applications, as well as in applications wherein stenting has not been successful due to limitation of current stents and stent delivery systems. For example, the pleated stent assembly may be used in conventional coronary applications. Alternatively, in a preferred embodiment, the pleated stent assembly of the present invention is used to treat aneurysms. The pleated stent assembly of the present invention can be used to treat most aneurysms, including berry, or saccular, aneurysms and fusiform aneurysms located in neurovascular arteries, in the abdominal aortic artery and other arteries.

When used to treat aneurysms, the stent of the present invention preferably comprises a substantially solid body section between two expandable anchor sections. The pleated stent assembly of the present invention can be used to treat neurovascular aneurysms by providing the required combination of (i) flexibility for delivery, (ii) a sufficiently dense wall to cover the aneurysm and exclude blood circulation in the aneurysm and (iii) the ability to properly size the placed stent to fix its location without damage to the artery, a combination not found in currently available stents.

When used to treat an aneurysm, the stent of the present invention is positioned in an artery at the point of the aneurysm, such that the substantially solid body section of the wall of the stent covers the aneurysm, thereby blocking blood flow to the aneurysm to induce thrombosis in the aneurysm, promote healing and reduce risk of rupture. Deprived of blood circulation, the material in the aneurysm will solidify and the volume of the aneurysm will gradually reduce in volume. Additionally, the lattice-like struts forming the wall of the stent will serve as a platform for growth of new tissue that will bridge the aneurysm, forming a new natural wall for the vessel as the healing process progresses.

The use of a stent to treat aneurysms in this manner was not possible with prior technology. Use of the pleated stent assembly of the present invention allows the area of the stent that bridges the aneurysm to be solid or nearly solid, thereby excluding the aneurysm from the circulation without the need for coils. Additionally, since the anchor sections of the stent may be balloon expanded, the final placement diameter of the stent may be fine-tuned based on visual angiographic feedback. As a result, appropriate contact between the stent and interior wall of the vessel can be achieved. The stent may be patterned to provide longitudinal flexibility, and since non-compliant balloons are not required, elastic or semi-compliant balloons can be used to improve the longitudinal flexibility of the assembly. As a result of the flexibility of the pleated stent assembly of the present invention, the stent can be tracked through the carotid siphon and placed distal to the carotid siphon. The pleated stent can be used with difficult-to-treat aneurysms of the internal carotid or the basilar artery without most of the drawbacks of existing treatment methods. In cases of aneurysms in skull base locations that are very difficult to reach surgically, use of the pleated stent assembly of the present invention promises to be more effective and safer than prior surgery or coiling methods.

### BRIEF DESCRIPTION OF THE DRAWINGS

FIG. 1 is a perspective view of one embodiment of a stent for use with the pleated stent assembly of the present invention.

FIGS. 2A, B, C and D depict steps in forming the pleated stent assembly of the present invention.

FIG. 3 is a plan view of the wall pattern of the stent of FIG. 1.

FIG. 4 is a cross-sectional view of a stent of the present invention positioned within a body vessel.

### DETAILED DESCRIPTION OF PREFERRED EMBODIMENT

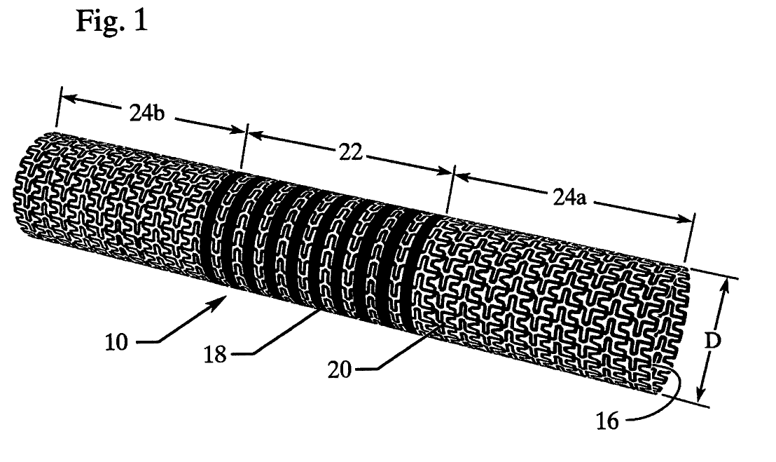

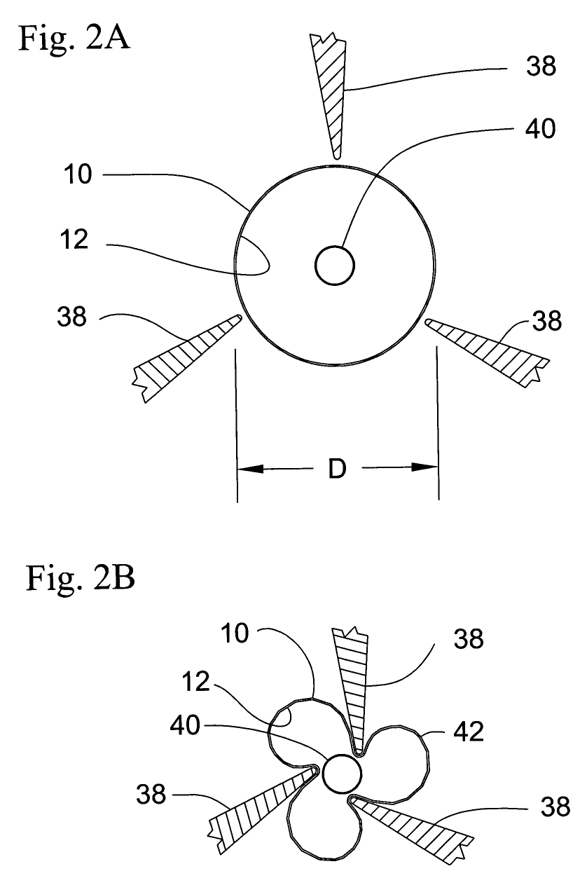

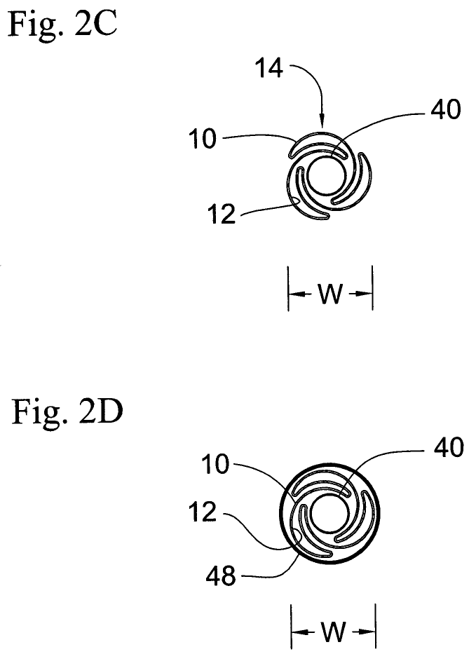

The present invention is directed to a pleated medical device assembly comprising a tube co-pleated with a balloon. In the preferred embodiment depicted in the drawings, the medical device is a stent 10, as best shown in FIG. 1. Turning to FIGS. 2A, B and C, stent 10 is co-pleated with balloon 12 to form pleated stent assembly 14. Pleated stent assembly 14 comprises stent 10, having original diameter D, and balloon 12, wherein at least a portion of balloon 12 is contained within stent 10. Preferably, balloon 12 extends through the entire length of stent 10. Stent 10 is co-pleated with balloon 12 along longitudinal pleating lines to form a substantially cylindrical pleated tube/balloon assembly 14, having a delivery width W, less than original diameter D, and suitable for intraluminal delivery. Stent assembly 14 may be delivered to the target location in a body vessel using standard techniques well know in the art.

Once positioned at the desired target location within the vessel, pressure within balloon 12 is increased to simultaneously unfold stent 10 and balloon 12 within the vessel. Pressure within balloon 12 may be further increased to expand at least a portion of stent 10 until at least a portion of the exterior surface of stent 10 presses against the interior of the vessel to hold stent 10 in place within the vessel. Balloon 12 is then deflated and removed from stent 10 and the vessel.

Returning to FIG. 1, in the preferred embodiment, stent 10 comprises tubular wall 16, manufactured with original diameter, D. Original diameter D refers to the diameter of stent 10 prior to pleating with balloon 12. Original diameter D is preferably the as-manufactured diameter, but may be a diameter larger or smaller than the manufactured diameter. Original diameter D is substantially equal to the final desired placement diameter of stent 10 within the vessel. Preferably, original diameter D is slightly less than the diameter of the vessel into which stent 10 will be delivered. For example, for stents of the present invention used to treat neurovascular aneurysms (“neurovascular stents”), original diameter D is generally between about 2.0 mm and 4.0 mm. For stents of the present invention used to treat abdominal aortic aneurysms (“AAA stents”), original diameter D is generally about 25 mm. For coronary stents of the present invention, original diameter D is generally between about 2.0 mm and 4.0 mm. However, the diameter will vary based on the particular vessel and application. Diagnostic imaging can be used to determine the appropriate diameter and length of stent 10.

Wall 16 of stent 10 is formed of a biocompatible material sufficiently ductile to accommodate pleating and unpleating without tearing. The biocompatible material is preferably a biocompatible metal or plastic. The more ductile the material used to form wall 16, the thicker it may be. Preferably, wall 16 is comprised of pure gold. In such embodiment, wherein wall 16 is formed of a ductile material, wall 16 may have a thickness up to about 0.003 inches, preferably about 0.001 inches, for neurovascular and coronary stents. Stents formed of less ductile materials must be thinner to accommodate pleating and unpleating without tearing. The capability of stent 10 to contain a large percentage of solid area also allows for a thin wall 16 that provides radial strength equivalent to stents with much thicker walls. Thinner walled stents will take up less cross sectional area in the vessel, resulting in a bigger lumen. The thickness and material of wall 16 may be tailored to provide a stent optimized for a particular stent application. Exemplary thicknesses of stents for use with the pleated stent assembly of the present invention made from ductile electroformed gold include the following: AAA stents will be thicker than neurovascular stents. Generally, AAA stents would be about 0.003 inch think and neurovascular stents would be about 0.0005 inch to 0.001 inch think. Coronary stents would typically be about 0.001 inch thick. Of course, the thickness will vary based on the specific application.

As depicted in FIG. 1, wall 16 of stent 10 is preferably comprised of a pattern of interconnected solid areas 18 defining open spaces 20 therebetween. Open spaces 20 provide the longitudinal flexibility necessary for delivery and facilitate stent 10 being covered with, and imbedded in, new body tissue.

In the embodiment of stent 10 depicted in FIG. 1, suitable for treatment of aneurysms, the pattern of wall 16 comprises annular body section 22 and annular anchor sections 24a and b. Preferably, body section 22 of wall 16 is solid, or substantially solid, and is not radially expandable substantially beyond original diameter D of stent 10. However, some expansion capability may be designed into body section 22. Preferably, anchor sections 24a and b of wall 16 are expandable beyond original diameter D. The pattern of anchor sections 24a and b may be designed to expanded further for a given balloon pressure. When used herein, “expandable” means expandable by balloon 12 under standard conditions.

Stent 10 having both body section 22 and anchor sections 24a and b allows for anchoring stent 10 by radial expansion of anchor sections 24a and b. When stent 10 is an aneurysm-treating stent, body section 22 preferably is longer than the aneurysm being treated, so that anchor sections 24a and b will be expanded into the sound sections of the artery proximal and distal to the aneurysm.

For stent 10 of FIG. 1, suitable for treatment of aneurysms, body section 22 of wall 16 comprises at least about 40 percent solid area, more preferably between about 50 and 100 percent, and most preferably between 80 and 95 percent solid area. In such embodiment, the purpose of body section 22 is to restrict blood circulation in the aneurysm. Therefore, open spaces 20 in body section 22 will be small, preferably less than about 50 microns wide. Stents for aneurysms distal to the carotid siphon, i.e. neurovascular stents, will require more flexibility than stents for more easily reached aneurysms, i.e., AAA stents. For neurovascular stents, wall 16 of body section 22 will preferably comprise about 60 percent or greater solid area, and more preferably between about 70 and 85 percent. For AAA stents, wall 16 of body section 22 preferably will comprise about 70 percent or greater solid area, and more preferably will be between about 75 and 90 percent solid.

The pattern of anchor sections 24a and b of wall 16 of stent 10 is designed to provide the flexibility necessary for delivery and radial expansion, and the radial strength necessary to prevent stent 10 from moving relative to the artery after placement. Thus, anchor sections 24a and b do not require a large solid area. Preferably the anchor section comprises less than about 50% solid area. Anchor sections 24a and b of aneurysm-treating stents require very little radial expansion since the original diameter D is selected to be just slightly less than the diameter of the artery. Anchor sections 24a and b of aneurysm-treating stents will typically be designed to expand about 0 to 30 percent beyond the original diameter D. Actual expansion will be limited to the amount needed to properly seat anchor sections 24a and b against the artery wall.

In alternative embodiments, wall 16 of stent 10 may contain one or more anchor sections 24 and one or more body sections 18, which may be arranged in any order along the length of stent 10. Alternatively, stent 10 may comprise only body section 22 or anchor section 24. If body section 22 is not present, and the entire length of stent 10 is capable of expansion as an elongated anchor section 24, the pleated stent can be used as a direct replacement for most current stent applications, including coronary stents. The pattern of such stent could be designed to accommodate a large expansion. For example, a pattern similar to typical balloon expandable coronary stents could be used, and would allow expansion to more than 300 percent of the original diameter D. Such stent patterns would provide a very small percent of metal against the vessel wall. It should be understood that stent 10 used in the pleated stent assembly of the present invention can be configured in a wide variety of patterns. The specific pattern required will vary depending on the application, as can be determined by one in the art.

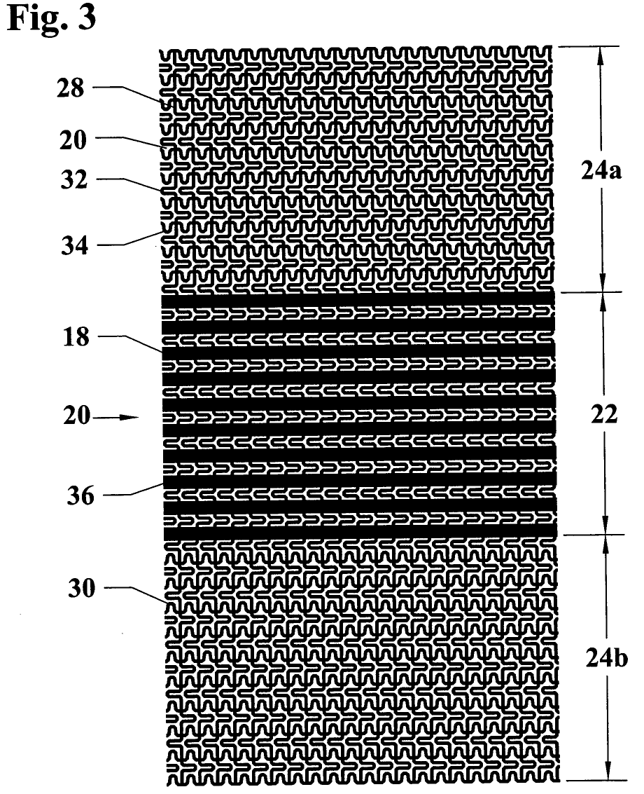

Turning to FIG. 3, pattern 26 is appropriate for stent 10 configured for the treatment of neurovascular aneurysms. Pattern 26 is designed to restrict radial expansion within body section 22 and to allow radial expansion in anchor sections 24a and b. Pattern 26 comprises longitudinal struts 28, which extend along the length of stent 10, and interconnected circumferential struts 30, which extend around the circumference of stent 10. Preferably longitudinal struts 28 contain one or more longitudinal loops 32 to allow longitudinal flexibility for delivery. Circumferential struts 30 in anchor sections 24a and b are radially expandable beyond original diameter D of stent 10. In a preferred embodiment, circumferential struts 30 in anchor section 24 contain at least one circumferential loop 34 to allow circumferential expansion.

Circumferential struts 30 of body section 22 are radially non-expandable substantially beyond original diameter D of stent 10. In a preferred embodiment, circumferential struts 30 of body section 22 are wider than longitudinal struts 28 and circumferential struts 30 of anchor sections 24a and b. In the most preferred embodiment, circumferential struts 30 of body section 22 are wider circumferential bands 36, with no circumferential loops, and longitudinal struts 28 allow longitudinal flexibility. It should be understood that pattern 26 is an exemplary preferred pattern, and countless other patterns may be used within the present invention and would fall within the scope of the claims.

Stent 10 of the present invention may be formed by any process capable of forming the desired stent pattern. In the preferred embodiment, stent 10 is formed by electroforming, as described in U.S. Pat. Nos. 6,019,784 and 6,274,294 and U.S. patent application Ser. No. 10/452,891, which are hereby incorporated by reference. In the electroforming process, the desired pattern is defmed by a photoresist exposed on a sacrificial mandrel. The electroforming process essentially grows stent 10 from the sacrificial mandrel to any desired thickness, after which the mandrel is dissolved. Unlike many other fabrication processes, stents having thin walls can be produced easily by electroforming.

In a preferred embodiment, gold electroformed stents for use in the pleated stent assembly of the present invention may be manufactured using cylindrical photolithography on a sacrificial mandrel, similar to that disclosed in U.S. Pat. Nos. 6,274,294 and 6,019,784, as follows:

An electrically conductive cylindrical mandrel, i.e., brass, copper, aluminum tube or wire is coated with photoresist by threading the mandrel through a hole in a rubber diaphragm at the bottom of a small cup holding liquid photoresist. With the mandrel held stationary in a vertical position, the cup is slowly pulled down the tube at an appropriate rate to coat the mandrel with an appropriate thickness of resist. A positive or a negative resist may be used. The liquid resist is soft baked to dry, but at a sufficiently low temperature not to destroy the photosensitivity of the resist.

The resist may be exposed with the stent image as described in U.S. Pat. Nos. 6,274,294 or 6,019,784. Because the stents of the present invention are made at or near the artery size, the mandrels used for neurovascular and coronary stents are typically larger than mandrels used for standard coronary stents that are made at or near the delivery diameter. Multiple stents may be imaged on a single mandrel.

The photoresist is developed in a developer solution appropriate for the resist used. The developed resist contains openings corresponding to the stent pattern, which exposes the surface of the mandrel. The developed resist can be hard baked to toughen it so that it survives the electroplating run.

The resist-coated mandrel is typically fitted with a conductive extension or stem. The extension is passed through or fitted with a slip ring for electrical contact. The mandrel is supported vertically so that the stent images are below the surface of the gold electroplating bath. The electroplating bath contains a platinum anode. Electrical current from a pulse-plating power supply is passed through the plating cell with the negative lead connected to the slip ring contact that is connected to the mandrel. The positive lead is connected to the anode. The mandrel is rotated about its axis during the plating run to maintain uniform stent strut thickness. The stent electroforming continues for a predetermined number of amp-minutes necessary to obtain the desired stent strut thickness. A porous gold electroformed layer may be formed as described in U.S. patent application Ser. No. 10/452,891.

Following electroplating of the stent and the optional porous layer, the mandrel is removed from the plating bath and rinsed. The photoresist is stripped in an appropriate solution to expose the mandrel. The copper, brass or aluminum mandrel is then dissolved to free the stents. The completed stents are rinsed and dried.

A drug or drugs may optionally be loaded into the porous layer or coated directly on the stent. Stents patterned to have walls with a large percentage of solid area provide a good platform for drug delivery by providing a large area to carry the drug and by improving the uniformity of drug delivery.

Stent 10 can alternatively be formed by any means known in the art or hereafter developed. For example, thin-walled cylindrical tubes may be formed on a cylinder by electroplating, vacuum evaporation or sputtering. The thin-walled tube thus formed can be patterned using cylindrical photolithography and etching of the unprotected material. The mandrel can then be dissolved to free the stent. Alternatively, the thin walled tubes can be photolithographically patterned and chemically etched or electro-etched to form the desired pattern in the tube, or the stent can be machined or laser machined to form the desired pattern.

Once formed, stent 10 is pleated onto balloon 12 to form pleated balloon assembly 14, as shown in FIGS. 2A, B and C. Preferably, stent 10 and balloon 12 are co-pleated by first placing at least a portion of balloon 12 within stent 10. Balloon 12 is preferably a plastic or rubber angioplasty balloon in a standard balloon catheter configuration. A balloon having an as-molded diameter that is essentially equal to original diameter D of stent 10 is preferred.

The type of balloon used will depend on the application. For example, when stent 10 is an aneurysm-treating stent and comprises both body section 22 and anchor sections 24a and b, balloon 12 is preferably a semi-compliant or elastic balloon, able to expand the stent to a larger diameter at anchor sections 24a and b without damaging the essentially non-expandable body section 22. Neurovascular stents require a balloon 12 having a thin and flexible wall. When stent 10 is used in a coronary application, requiring an essentially cylindrical expansion along the length of the stent, balloon 12 is preferably a non-compliant balloon.

To co-pleat balloon 12 and stent 10, balloon 12 is expanded at a low pressure to bring the exterior surface of balloon 12 into contact with the interior surface of stent 10, as shown in FIG. 2A. While maintaining the pressure in the balloon 12 at a constant value, heated longitudinal blades 38 are advanced in a radial or nearly radial direction toward the longitudinal axis of stent 10 and balloon 12, until blades 38 contact stent 10 along equally spaced longitudinal pleating lines. Preferably three equally spaced longitudinal pleating lines are used, although one or more pleating lines could be used consistent with the present invention. With the longitudinal axis of stent 10 and balloon 12 centered between blades 38, blades 38 are advanced toward the longitudinal axis, near the central lumen 40 of the balloon catheter, to form lobes 42, as depicted in FIG. 2B.

A temperature high enough to set the pleated shape in balloon 12 is maintained. Pressure in balloon 12 is released and blades 38 are then retracted. Lobes 42 are rolled onto central lumen 40 of the balloon catheter, forming a substantially cylindrical pleated tube/balloon assembly 14 having delivery width W, suitable for intraluminal delivery. Delivery width W of tube/balloon assembly 14 is preferably about ⅓ to ¼ of the original diameter D of stent 10. Preferably stent 10 is formed from a material that undergoes sufficient plastic deformation along the longitudinal pleating lines to substantially maintain the delivery width W of tube/balloon assembly 14.

Heat may be used to set the final pleats in balloon 12, to help ensure its ultimate clean removal from the delivered stent 10. After delivery of stent 10, the memory of the heat-set pleats will pull deflated balloon 12 to a smaller configuration so that it can be removed from stent 10 without snagging stent 10.

Commercial balloon pleating fixtures may be used to pleat the stent/balloon assembly 14. For example, Interface Associates in Laguna Niguel, California sells a Fluting Fixture, 3F/4F/6F-300 that can be used.

Pleated stent assembly 14 is delivered to the desired location by first inserting pleated stent assembly 14 into the appropriate vessel in the body of a subject using conventional methods. The subject may be a human or other animal. It should be understood that the pleated stent assembly of the present invention may be inserted and delivered into arteries, other types of vessels and other organs having a lumen. As used herein, the term “vessel” includes any vessel or other organ having a lumen, unless otherwise specified.

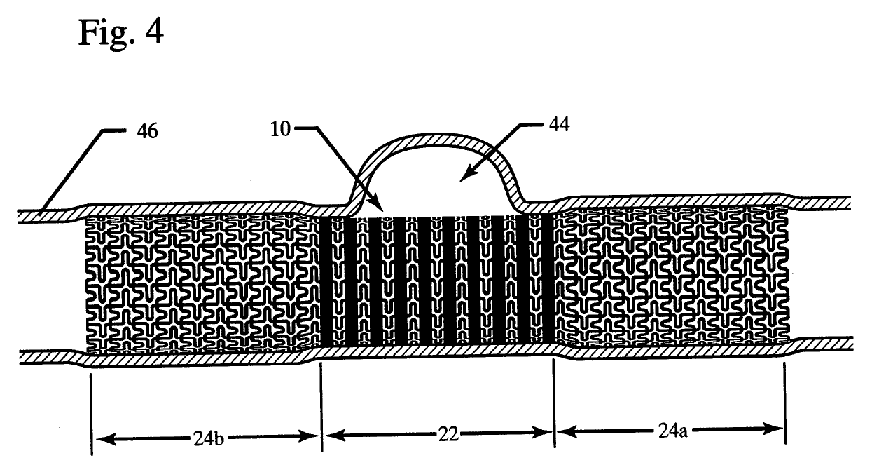

Pleated stent assembly 14 is advanced to a desired position within the artery using a guide wire. In one preferred embodiment depicted in FIG. 4, pleated stent assembly 14 is used to treat an aneurysm, and pleated stent assembly 14 is delivered to a location in artery 46 adjacent aneurysm 44. In such embodiment, pleated stent assembly 14 is centered on aneurysm 44, based on angiographic imaging using iodine to contrast the artery 46 and aneurysm 44 from the background. The radiopacity of the gold stent aids in positioning the body section 22 of stent 10 over the aneurysm.

Once in position within the artery, the pressure within balloon 12 is increased to simultaneously unfold stent 10 and balloon 12 until stent 10 and balloon 12 are fully unpleated to their original cylindrical shape. In the case of a neurovascular stent, the pressure in the semi-compliant balloon is increased to approximately 0.5 atmospheres to unpleat balloon 12 and stent 10. Higher expansion pressures are used to unpleat a coronary stent and balloon in order to push back the material responsible for the stenosis and form a substantially cylindrical lumen in the vessel.

In the embodiment wherein stent 10 comprises body section 22 and anchor sections 24a and b, once stent 10 and balloon 12 are fully unpleated, the pressure within balloon 12 is further increased to expand anchor sections 24a and b of stent 10 beyond original diameter D of stent 10, until the exterior surface of anchor sections 24a and b press against the wall of the vessel. The pressure within balloon 12 is increased based on visual angiographic feedback to provide the optimum amount of expansion. Optimum expansion will seat anchor sections 24a and b in the artery wall with little or no expansion of the artery to minimize damage to the artery, while securing stent 10 in place. This will typically require expansion of the anchor sections between about 0 and 30 percent beyond of the original diameter D. For a neurovascular stent, increasing the pressure from approximately 0.5 atm to 2 atm. will increase the diameter of the anchor sections 24a and b approximately 20 percent.

Once stent 10 is seated in the aneurysm, the pressure within balloon 12 is decreased to evacuate balloon 12 and balloon 12 is removed from stent 10, the vessel and the body. The plastic deformation that occurs during the radial expansion causes any expanded sections of stent 10 to remain expanded after removal of balloon 12.

In the embodiment wherein stent 10 is a coronary stent, stent 10 at original diameter D is patterned similar to a conventional balloon-expandable coronary stent at its expanded placement diameter, i.e. with a 12 to 20 percent solid surface. In such embodiment, stent 10 would be formed from a ductile material, preferably gold, to accommodate pleating and unpleating without tearing. Such stent would be used in coronary applications, with a non-compliant balloon. In such embodiment, stent 10 would be unfolded with balloon 12 within the artery and could be further expanded by increasing the pressure in balloon 12 to seat stent 10 in the artery, resulting in a uniformly expanded stent.

In yet another embodiment, stent 10 is a self-expanding stent is formed from an elastic or super-elastic material. In such embodiment, the pleated geometry can be maintained by a cylindrical sleeve 48 placed over stent 10, as depicted in FIG. 2D. Sleeve 48 will be capable of being pulled away from stent 10 after delivery, allowing stent 10 to self expand within the vessel. In such embodiment, stent 10 may be formed with anchor section 24 to allow balloon expansion, after self-expansion, to maintain stent 10 in place within the vessel.

The pleated stent assembly of the present invention can be used in a wide variety of applications. For example, aneurysms are often located at points of bifurcation in the artery. Two or three stents may be used to treat aneurysms associated with a bifurcation. Three stents are used if the aneurysm involves the area proximal to the bifurcation. If three stents are needed to cover the aneurysm, one large stent and two smaller stents are used. For neurovascular stents, the larger stent is first placed in the proximal section of the artery. The larger stent consists of only two sections, a nonexpanding distal body section and a proximal expanding anchor section. Smaller stents are then delivered into each leg of the bifurcation using “kissing” balloons, i.e., two side by side balloons on separate guide wires. The smaller stents have expandable anchor sections on both ends and a nonexpanding central body section. The proximal expanding anchor sections would be located within the length of the larger stent. The nearly solid body sections of the three stents would combine to form a bifurcated vessel that spans the aneurysm. For AAA stents, because access to the abdominal aorta is from the femoral arteries, the larger stent would be placed first, and both the left and the right femoral arteries would provide access to place the two smaller kissing stents that would be nested inside the larger first stent placed in the aorta.

Two smaller stents can be used for the more common case wherein the aneurysm is located at the vertex of a bifurcation and only involves the two branch arteries. Each of the two smaller stents would have an expanding distal anchor section and a non expanding proximal body section. The non-expanding proximal body section of each stent would span the aneurysm, and the expanding distal anchor sections would anchor one stent in each of the branches. The two stents could be delivered simultaneously with kissing balloons or sequentially. The wide range of variations in artery and aneurysm geometry at a bifurcation will require customizing the technique to best fit the situation. Therefore it should be understood that the above is only representative of a general approach.

From the foregoing it will be seen that this invention is one well adapted to other advantages which are obvious and which are inherent to the invention. For example, it should be understood that the pleated stent assembly of the present invention can be used for other applications and to treat other types of aneurysms and vascular conditions. In addition to use with stents, the pleated medical device assembly can be used with other tubular medical devices. Further, when used herein, “medical device” is meant to refer to medical devices used to treat humans and veterinary devices used to treat animals.

Since many possible embodiments may be made of the invention without departing from the scope thereof, is to be understood that all matters herein set forth or shown in the accompanying drawings are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and sub-combinations are of utility and may be employed without reference to other features and sub-combinations. This is contemplated by and is within the scope of the claims.

1. A pleated stent assembly comprising:
a balloon; and
a tube having an original diameter,
wherein at least a portion of said balloon is contained within said tube,
wherein said tube and said balloon are co-pleated along longitudinal pleating lines to form a substantially cylindrical pleated tube/balloon assembly having a delivery width, and
wherein said delivery width of said assembly is less than said original diameter of said tube.

2. The device of claim 1, wherein said tube is formed from a material that undergoes sufficient plastic deformation along said pleating lines to substantially maintain said delivery width of said tube/balloon assembly.

3. The device of claim 1, further comprising a tubular sleeve substantially surrounding said tube/balloon assembly to substantially maintain said delivery width of said tube/balloon assembly.

4. The device of claim 3 wherein said tube is formed from a material having super-elastic properties.

5. The device of claim 1, wherein said tube is flexible along its longitudinal axis.

6. The device of claim 1, wherein the wall of said tube comprises at least one substantially solid annular body section.

7. The device of claim 6, wherein said body section is not radially expandable substantially beyond said original diameter upon inflation of said balloon.

8. The device of claim 1, wherein the wall of said tube comprises at least one annular anchor section, wherein said anchor section is radially expandable beyond said original diameter upon inflation of said balloon.

9. The device of claim 7, wherein the wall of said tube comprises at least one annular anchor section, wherein said anchor section is radially expandable beyond said original diameter upon inflation of said balloon.

10. The device of claim 1, wherein the wall of said tube is comprised of a pattern of interconnected solid areas defining open spaces therebetween.

11. The device of claim 10, wherein said pattern restricts radial expansion of said tube substantially beyond said original diameter over a portion of the length of said tube.

12. The device of claim 11, wherein said pattern comprises greater than about 60 percent solid area in the portion of said tube wherein radial expansion is restricted.

13. The device of claim 11, wherein said pattern allows radial expansion of said tube beyond said original diameter over at least a portion of the length of said tube.

14. The device of claim 13, wherein said pattern allows radial expansion up to about 130% of said original diameter in the portion of said tube wherein radial expansion is allowed.

15. The device of claim 10, wherein said solid areas are comprised of longitudinal struts and interconnected circumferential struts.

16. The device of claim 1 5, wherein said wall comprises at least one annular anchor section, wherein the circumferential struts in said anchor section are radially expandable beyond said original diameter.

17. The device of claim 16, wherein said wall comprises at least one annular body section, wherein the circumferential struts in said body section of said wall are radially non-expandable substantially beyond said original diameter.

18. The device of claim 1, wherein said tube is a stent.

19. The device of claim 18, wherein said tube is formed from an electroformed metal.

20. The device of claim 19, wherein said metal is gold.

21. The device of claim 1, wherein said tube is formed from a biocompatible plastic.

22. A medical or veterinary stent comprising:
a tubular wall,
wherein said wall comprises at least one annular body section and at least one annular anchor section,
wherein said body section is substantially non-expandable radially, and
wherein said anchor section is expandable radially.

23. The stent of claim 22, wherein said anchor section is expandable up to about 130% of its original diameter.

24. The stent of claim 22, wherein said wall is comprised of a pattern of interconnected solid areas defining open spaces therebetween, and wherein the pattern of said body section of said wall comprises at least about 80% solid area and wherein the pattern of said anchor section of said wall comprises less than about 50% solid area.

25. The stent of claim 24, wherein said solid areas of said wall are formed from an electroformed metal.

26. The stent of claim 25, wherein said metal is gold.

27. A method for delivering a pleated stent assembly comprising:
obtaining a pleated stent assembly comprising a stent longitudinally pleated onto and with a balloon;
inserting said pleated stent assembly into a vessel of a subject;
advancing said pleated stent assembly to a desired position within the vessel;
increasing the pressure within the balloon to simultaneously unfold the stent and balloon until the stent and balloon are fully unpleated;
decreasing the pressure within the balloon; and
removing the balloon from the stent and the vessel.

28. The method of claim 27, further comprising after said increasing step, the step of further increasing the pressure within the balloon to expand at least a portion of said stent until at least a portion of the exterior surface of said stent presses against the interior of said vessel.

29. The method of claim 28, wherein said stent comprises at least one non-expandable body section, and at least one expandable anchor section, wherein said further increasing step comprises expanding the anchor section of said stent until the exterior surface of said anchor section presses against the interior of said vessel.

30. The method of claim 29, wherein said vessel is an artery and said desired position is adjacent to an aneurysm.

31. A method for forming a pleated stent assembly comprising:
inserting at least a portion of a balloon within a tube having an original diameter; and
co-pleating said balloon and said tube along longitudinal pleating lines to form a substantially cylindrical pleated tube/balloon assembly having a delivery width,
wherein said delivery width of said assembly is less than said original diameter of said tube.
